(19) United States
(12) Patent Application Publication
(10) Pub. No.: US 2024/0296608 A1
(43) Pub. Date: Sep. 5, 2024

(54) Rendering Tool Information As Graphic Overlays On Displayed Images Of Tools

(71) Applicant: INTUITIVE SURGICAL OPERATIONS, INC., Sunnyvale, CA (US)

(72) Inventors: Brandon D. Itkowitz, San Jose, CA (US); Simon P. DiMaio, San Carlos, CA (US); Daniel J. Halabe, Los Altos, CA (US); Christopher J. Hasser, Los Altos, CA (US); Brian David Hoffman, Mountain View, CA (US); David Q. Larkin, Menlo Park, CA (US); Catherine J. Mohr, Mountain View, CA (US); Paul W. Mohr, Mountain View, CA (US); Tao Zhao, Sunnyvale, CA (US); Wenyi Zhao, Weston, FL (US)

(21) Appl. No.: 18/437,072

(22) Filed: Feb. 8, 2024

Related U.S. Application Data
(63) Continuation of application No. 17/213,924, filed on Mar. 26, 2021, now Pat. No. 11,941,734.
Application No. 17/213,924 is a continuation of application No. 16/351,444, filed on Mar. 12, 2019, now Pat. No. 10,984,567.
Application No. 16/351,444 is a continuation of application No. 15/946,408, filed on Apr. 5, 2018, now Pat. No. 10,282,881.
Application No. 15/946,408 is a continuation of application No. 13/768,047, filed on Feb. 15, 2013, now Pat. No. 10,008,017.
Application No. 13/768,047 is a continuation-in-part of application No. 12/415,354, filed on Mar. 31, 2009, now Pat. No. 9,789,608.

(51) Int. Cl. G06T 11/60; A61B 34/00; A61B 34/20; A61B 34/30; A61B 34/37; A61B 90/00; B25J 9/16; G05B 19/42; G06T 11/40

(52) U.S. Cl. CPC G06T 11/60; A61B 34/30; A61B 34/37; A61B 90/36; B25J 9/1666; B25J 9/1671; B25J 9/1692; G05B 19/4202; G06T 11/40; A61B 34/20; A61B 2034/2061; A61B 34/25; A61B 90/361; A61B 2090/371; G05B 2219/36432; G05B 2219/39083; G05B 2219/39096; G05B 2219/39449; G05B 2219/40607; G05B 2219/45117; G05B 2219/45123; Y02P 90/02

(57) ABSTRACT

A system comprises: a robotic arm operatively coupleable to a tool comprising a working end; and an input device communicatively coupled to the robotic arm. The input device is manipulatable by an operator. The system further comprises a processor configured to cause an image of a work site, captured by an image capture device from a perspective of an image reference frame, to be displayed on a display. The image of the work site includes an image of the working end of the tool. The processor is further configured to determine a position of the working end of the tool in the image of the work site and render a tool information overlay at the position of the working end of the tool in the image of the work site. The tool information overlay visually indicates an identity of the input device.

### CROSS REFERENCE TO RELATED APPLICATIONS

This application is a continuation of U.S. application Ser. No. 15/946,408 (filed Apr. 5, 2018), which is a continuation of U.S. application Ser. No. 13/768,047 (filed Feb. 15, 2013), now U.S. Pat. No. 10,008,017, which is a continuation-in-part of U.S. application Ser. No. 12/415,354 (filed Mar. 31, 2009), now U.S. Pat. No. 9,789,608, each of which is incorporated herein by reference.

### FIELD OF THE INVENTION

The present invention generally relates to robotic systems and in particular, to a robotic system, and a method implemented therein, for rendering tool information as graphic overlays on displayed images of tools.

### BACKGROUND

In a robotic system, one or more tools may be telerobotically controlled by an operator to perform a procedure on an object at a work site. A camera is provided at the work site to capture images of end effectors of the tools as they interact with the object to perform the procedure, so that the operator may view their movements on a display while telerobotically controlling the tools using associated input devices.

During the procedure, it may be useful to provide the operator with tool information such as whether a tool is energized at the time or which of a plurality of tools is energized at the time or which robotic arm is operatively coupled to a specific tool at the time. The tool information may be provided as text or a graphic in an area on the display which is proximate to its corresponding tool so that the information may be readily associated with the tool. However, such positioning of the tool information may objectionably obstruct images of the tools and/or objects upon which the tools are performing a procedure at the time. Also, when the end effectors of two or more tools are in close proximity to one another, it may not be readily apparent which tool the tool information pertains to at the time. This may be especially problematic if the tool information is stationary and the end effectors are moving.

Alternatively, the tool information may be provided in an area that is not proximate to the tool, such as in a boundary area circumscribing the display viewing area or off to one side of the viewing area to avoid obscuring images of the end effectors and objects upon which the end effectors are performing a procedure at the time. When the tool information is provided outside the gaze area of the operator, however, it may be distracting for the operator to visually find and/or associate the provided tool information with its corresponding tool because the operator's eyes must shift from the area in which the operator is currently gazing to another area on the display. In the case of a stereo display, the situation becomes even more complicated, because the operator's eyes not only have to shift vertically and horizontally around the display to find the tool information, they may also have to look for and focus on tool information at a different depth than the three-dimensional images of the object and tools that the operator is viewing at the time on a stereo vision display.

### BRIEF SUMMARY

The embodiments of the invention are summarized by the claims that follow below.

### BRIEF DESCRIPTION OF THE DRAWINGS

FIG. 1 illustrates a top view of an operating room employing a robotic system utilizing aspects of the present invention.

FIG. 2 illustrates a front view of a movable cart usable in a robotic system utilizing aspects of the present invention.

FIG. 3 illustrates a perspective view of a tool usable in a robotic system utilizing aspects of the present invention.

FIG. 4 illustrates a front view of a console usable in a robotic system utilizing aspects of the present invention.

FIG. 5 illustrates a flow diagram of a method for providing tool information on a display utilizing aspects of the present invention.

FIG. 6 illustrates a simplified view of a display in which tool information is rendered over an image of a tool according to the method of FIG. 5.

FIG. 7 illustrates a simplified view of a display in which tool information is rendered over an image of a tool according to the method of FIG. 5.

### DETAILED DESCRIPTION

Although a medical robotic system is described herein, it is to be appreciated that the various aspects of the invention are not to be limited to medical robotic systems. They are applicable to robotic systems in general.

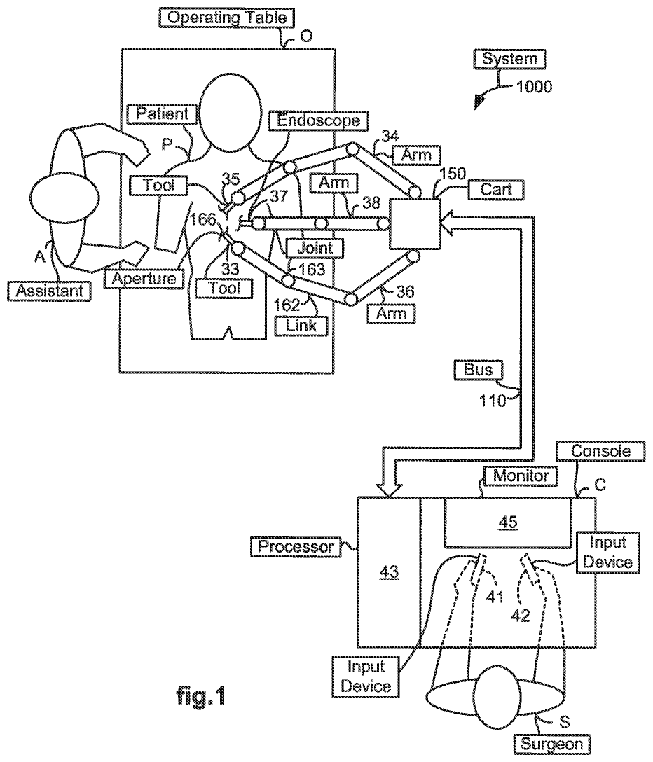

FIG. 1 illustrates a top view of an operating room in which a medical robotic system 1000 is being employed by a Surgeon (“S”) to perform a medical procedure on a Patient (“P”). The medical robotic system in this case is a Minimally Invasive Robotic Surgical (MIRS) system including a Console (“C”) utilized by the Surgeon while performing a minimally invasive diagnostic or surgical procedure on the Patient with assistance from one or more Assistants (“A”) while the Patient is on an Operating table (“O”).

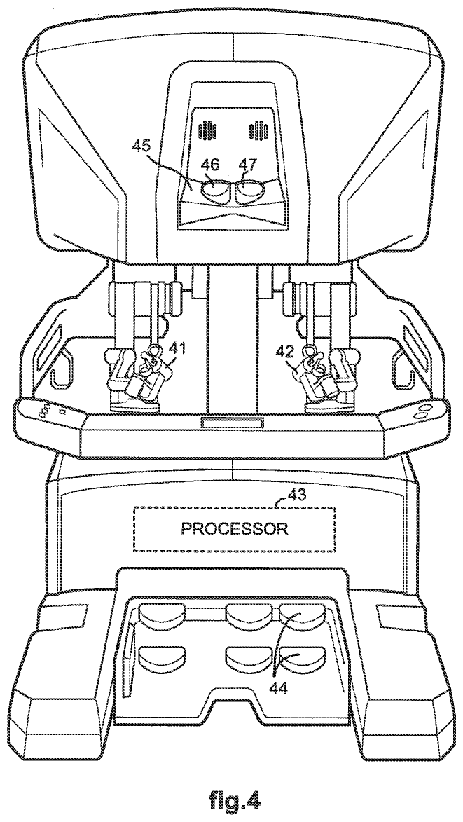

The Console, as further described in reference to FIG. 4, includes a processor 43 which communicates with a movable cart 150 over a bus 110. A plurality of robotic arms 34, 36, 38 are included on the cart 150. A tool 33 is held and manipulated by robotic arm 36, another tool 35 is held and manipulated by robotic arm 34, and an endoscope 37 is held and manipulated by robotic arm 38. In this example, each of the tools 33, 35 and the endoscope 37 is introduced through its own entry aperture in the Patient. As an example, tool 33 is inserted into aperture 166 to enter the Patient.

The Surgeon performs the medical procedure by manipulating the input devices 41, 42 so that the processor 43 causes their respectively associated robotic arms 34, 36 to manipulate their respective removably coupled tools 33, 35 accordingly while the Surgeon views real-time images of a work site in three-dimensions (“3D”) on a stereo vision display 45 of the Console. A stereoscopic endoscope 37 (having left and right cameras for capturing left and right stereo views) captures stereo images of the work site. The processor 43 processes the stereo images so that they may be properly displayed on the stereo vision display 45.

Each of the robotic arms 34, 36, 38 is conventionally formed of links, such as link 162, which are coupled together and manipulated through actuatable joints, such as joint 163. Each of the robotic arms includes a setup arm and a slave manipulator. The setup arm positions its held tool so that a pivot point occurs at its entry aperture into the Patient. The slave manipulator may then manipulate its held tool or endoscope so that it may be pivoted about the pivot point, inserted into and retracted out of the entry aperture, and rotated about its shaft axis. The robotic arms 34, 36, 38 may be carted into the operating room via the cart 150 or alternatively, they may be attached to sliders on a wall or ceiling of the operating room.

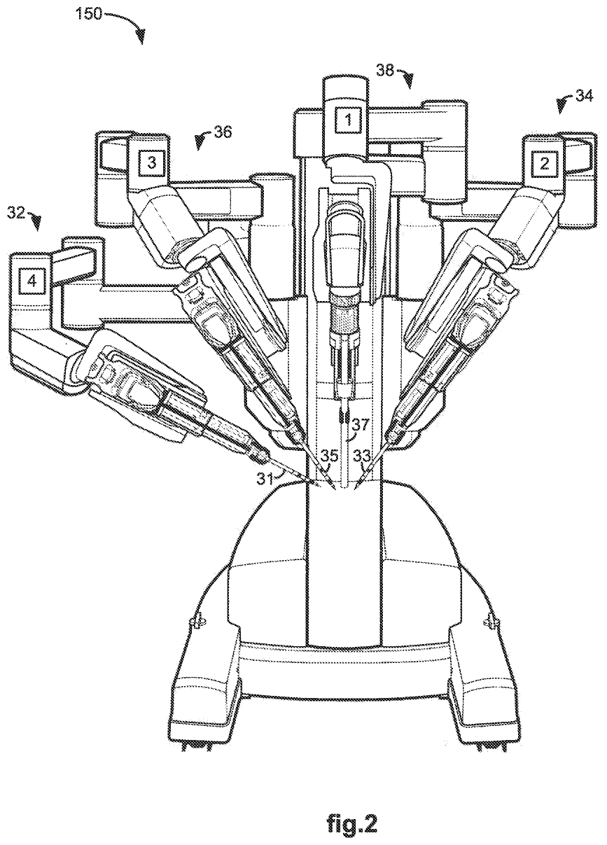

FIG. 2 illustrates a front view of the cart 150. In addition to the robotic arms 34, 36, 38, shown in FIG. 1, a fourth robotic arm 32 is shown in FIG. 2. The fourth robotic arm 32 is available so that another tool 31 may be introduced at the work site along with the tools 33, 35 and endoscope 37. Each of the robotic arms 32, 34, 36, 38 may be identified by a number as shown in FIG. 2 and/or a color.

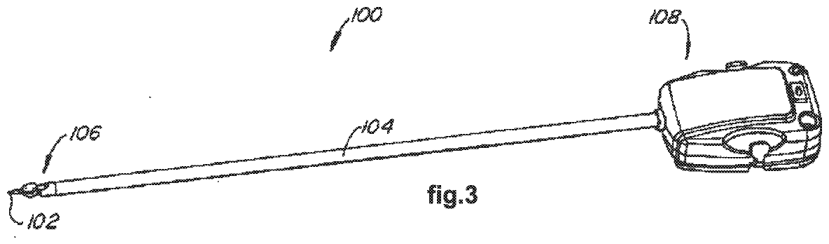

FIG. 3 illustrates an exemplary tool 100 that may be used for either tool 33 or 35. The tool 100 comprises an interface housing 108, a shaft 104, an end effector 102, and a wrist mechanism 106 which includes one or more wrist joints. The interface housing 108 is removably attached to a robotic arm so as to be mechanically coupled to actuators (such as motors) in the slave manipulator of the attached robotic arm. Cables or rods, that are coupled to the actuators of the slave manipulator and extend through the shaft 104 from the interface housing 108 to the one or more wrist joints of the wrist mechanism 106 and to the jaws of the tool's end effector 102, actuate the wrist joints and jaws in a conventional manner. The slave manipulator may also manipulate the tool in pitch and yaw angular rotations about its pivot point at the entry aperture, manipulate the tool in a roll angular rotation about the tool's shaft axis, and insert and retract the tool along a rail on the robotic arm as commanded by the processor 43.

FIG. 4 illustrates, as an example, a front view of the Console usable in the medical robotic system 1000. The Console has left and right input devices 41, 42 which the user may grasp respectively with his/her left and right hands to manipulate associated devices, such as the tools 33, 35, in preferably six degrees-of-freedom (“DOF”). Foot pedals 44 with toe and heel controls are provided on the Console so the user may control movement and/or actuation of devices associated with the foot pedals. A processor 43 is provided in the Console for control and other purposes. The stereo vision display 45 is provided so that the user may view the work site in stereo vision from images captured by the stereoscopic camera of the endoscope 37. Left and right eyepieces, 46 and 47, are provided in the stereo vision display 45 so that the user may view left and right two-dimensional (“2D”) display screens inside the display 45 respectively with the user's left and right eyes.

The processor 43 performs various functions in the medical robotic system. One important function that it performs is to translate and transfer the mechanical motion of input devices 41, 42 through control signals over bus 110 to command actuators of their associated robotic arms to actuate their respective joints so that the Surgeon can effectively manipulate devices, such as the tools 33, 35, and endoscope 37. Another function is to perform various methods described herein. Although described as a processor, it is to be appreciated that the processor 43 may be implemented by any combination of hardware, software and firmware. Also, its functions as described herein may be performed by one unit or divided up among different components, each of which may be implemented in turn by any combination of hardware, software and firmware. Further, although being shown as part of or being physically adjacent to the Console, the processor 43 may also comprise a number of subunits distributed throughout the system.

U.S. Pat. No. 6,659,939 B2 entitled “Cooperative Minimally Invasive Telesurgical System,” which is incorporated herein by reference, provides additional details on a medical robotic system such as described herein.

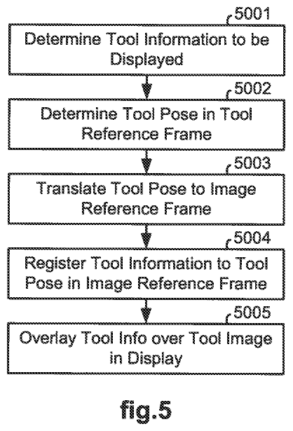

FIG. 5 illustrates a flow diagram of a method implemented by the processor 43 of the robotic system 1000 for providing tool information on the display 45. In block 5001, the method determines the information that is to be displayed for each tool operatively associated at the time with one of the input devices 41, 42. As an example, tool information may include information of which robotic arm each of the operative tools is operatively coupled to at the time. As another example, the tool information may include information of which tool is electrically active or energized at the time for cauterization or other purposes. The determination in this case may be made using information interactively provided using conventional means by an operator of the system and/or information pre-programmed into the system.

In block 5002, the method determines, for each tool which has tool information to be displayed, the current pose (i.e., position and orientation) of the tool in its tool reference frame. Each tool is operatively coupled to a robotic arm that manipulates the tool according to control commands generated by the processor 43 in response to operator manipulation of its associated input device. The manipulation of the tool is relative to a pivot point, which serves as origin for the tool reference frame. Determination of the current pose for each tool may be performed by using kinematics of the robotic arm and/or other well known techniques. Additional details may be found, for example, in U.S. 2006/0258938 A1 entitled “Methods and System for Performing 3-D Tool Tracking by Fusion of Sensor and/or Camera Derived Data during Minimally Invasive Robotic Surgery,” which is incorporated herein by reference.

In block 5003, for each tool which has tool information to be displayed, the method translates the determined tool pose in the tool reference frame to a tool pose in an image reference frame which is from the perspective of the stereo camera of the endoscope 37. As an example, the tool pose in the tool reference frame may first be translated to a tool pose in a fixed reference frame using a previously determined transform for the tool reference frame to the fixed reference. The tool pose in the fixed reference frame may then be translated to a tool pose in a camera reference frame using a previously determined transform from the fixed reference frame to the camera reference frame. Finally, the tool pose in the camera reference frame may be translated to a tool pose in the image reference frame using previously determined information of the camera pose in the camera reference frame. Additional details for such translations and transforms may be found, for example, in U.S. Pat. No. 6,424,885 entitled “Camera Referenced Control in a Minimally Invasive Surgical Apparatus”, which is incorporated herein by reference.

In block 5004, the method registers the tool information to the tool pose in the image reference frame. The tool information is preferably in the form of a three-dimensional overlay that conforms to the three-dimensional shape of the tool at a designated position on the tool. The tool information is then registered with the tool pose by registering the three-dimensional overlay at the designated position on the tool at the tool pose in the image reference frame. In the case where the tool information is a two-dimensional graphic of the tool information, a reference point of the two-dimensional graphic is registered to a designated point on the tool at the tool pose in the image reference frame.

In block 5005, the method renders the tool information as an overlay to the image of tool at the designated point in the display. In the case where the tool information is a three-dimensional overlay, rendering the overlay is straightforward. In this case, the rendering appears as a decal of the tool information which has been applied to the tool at the designated point. In the case where the tool information is a two-dimensional graphic, the two-dimensional graphic is bent to conform to the shape of the tool at the designated point and rendered so as to appear as if applying a decal of the tool information onto the tool at the designated point.

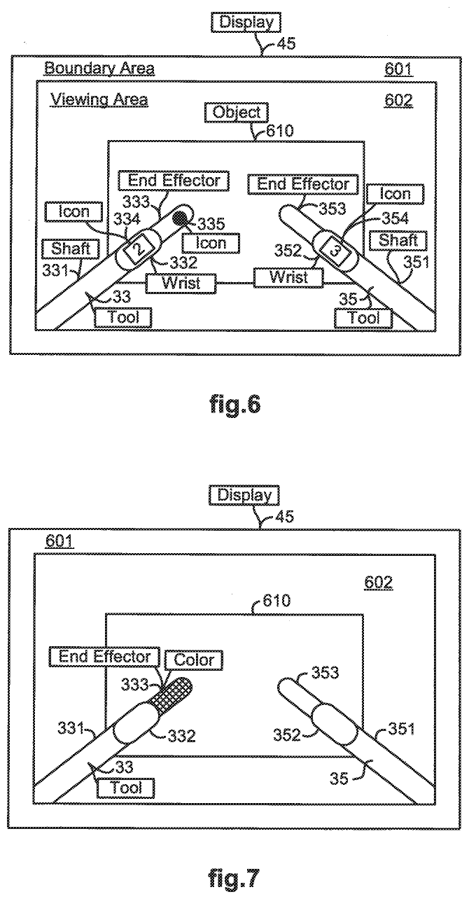

FIG. 6 illustrates, as an example, a simplified stereo view of the stereo vision display 45 from the perspective of an operator of the system 1000 after employing the method of FIG. 5 to render tool information in the form of icons 334, 335, 354 respectively over images of working ends of the tools 33, 35 on the display 45. A viewing area 602 displays images which have been processed to provide telepresence from images of the work site captured by the stereo camera of the endoscope 37. An optional boundary area 601 circumscribes the viewing area 602. In prior systems, tool information may be provided in the boundary area 601. The boundary area 601, however, is generally outside a current gaze area of the operator since the operator's eyes are focusing on images of end effectors 333, 353 of the tools 33, 35 on the display 45 as the end effectors 333, 353 interact with the object 610 at the work site to perform a procedure on the object 610.

The tool information for each tool, in this example, includes identification numbers of robotic arms which are operatively coupled to the tools 33, 35 at the time. In particular, the icon 334 has a numeral “2” on it to indicate the tool 33 is operatively coupled to the robotic arm 34, which is designated as robotic arm “2” by the numeral “2” being printed on it as shown in FIG. 2. Likewise, the icon 354 has a numeral “3” on it to indicate the tool 35 is operatively coupled to the robotic arm 36, which is designated as robotic arm “3” by the numeral “3” being printed on it as shown in FIG. 2. The icons 334, 354 are placed over images of wrists 332, 352 of their respective tools 33, 35. Alternatively, they may be overlayed over other designated points of the working ends of the tools 33, 35, such as on shafts 331, 351 or end effectors 333, 353.

Alternatively, or additionally, the tool information for each tool may indicate which input device and/or Surgeon is operatively associated at the time with the tool. When only one Surgeon is performing the procedure, then the letter “L” overlaid an image of the tool may indicate the tool is operatively associated with the left input device 41. Conversely, the letter “R” overlaid an image of the tool may indicate the tool is operatively associated with the right input device 42. When two or more Surgeons are performing the procedure, for example in collaboration using multiple consoles, then the initials of the Surgeon who is operatively associated with (i.e., has control of) the tool at the time may be overlaid an image of the tool. Alternatively, each Surgeon may be assigned a unique symbol or color which is overlaid the image of the tool that is being controlled by that Surgeon.

Alternatively, or additionally, the tool information for each tool may indicate state information for the tool, such as whether the tool is energized at the time or whether or not the tool is locked in position at the time. As an example, the mere presence of the icon 335 over the image of the end effector 333 may indicate tool 33 is energized at the time. Alternatively, an icon such as a red dot over the image of the end effector of a tool may indicate the tool is energized at the time. As another example, another icon such as a yellow dot over the image of the end effector of a tool may indicate the tool is locked in position at the time. Information of whether or not a tool is locked in position at the time is particularly useful for tools such as a cardiac stabilizer which is typically locked in position during a beating heart procedure so it cannot be inadvertently moved. The display of a locked or unlocked symbol over an image of the tool would serve as a reminder in this case to the Surgeon to lock the tool in place after positioning it.

Although static (non-moving) tool information is described herein, it is to be appreciated that the tool information may alternatively, or additionally, comprise dynamic or animated tool information when useful to better convey their meanings or draw the Surgeon's attention to them. For example, a pulsating red dot may be useful for quickly conveying to the Surgeon which tool is energized at the time to alert the Surgeon to avoid inadvertently touching its tip to unintended tissue areas. FIG. 7 illustrates a simplified stereo view of the stereo vision display 45 from the perspective of an operator of the system 1000 after employing the method of FIG. 5 to render tool information in the form of a color overlay over an image of the end effector 333 of the tool 33 on the display 45. The color overlay in this example indicates the tool 333 is being energized at the time for cauterization or other purposes. The color overlay may be any pre-designated color and remain over the image of the end effector either only temporarily (e.g., fading out) or during an entire period that the tool is being energized. Although the end effector is colored in this example to indicate that it is being energized, for example, with electrical or radio frequency power, other parts of the tool 33, such as its shaft or wrist, may be rendered with color instead or in addition to the end effector. Also, instead of using a color, a different brightness level over the image of the tool being energized may be used.

Although the various aspects of the present invention have been described with respect to a preferred embodiment, it will be understood that the invention is entitled to full protection within the full scope of the appended claims.

1-20. (canceled)

21. A system comprising:
a first robotic arm operatively coupleable to a first tool, the first tool comprising a working end;
a first input device communicatively coupled to the first robotic arm, the first input device being manipulatable by a first operator; and
a processor configured to:
cause an image of a work site, captured by an image capture device from a perspective of an image reference frame, to be displayed on a display, the image of the work site including an image of the working end of the first tool;
determine a position of the working end of the first tool in the image of the work site; and
render a first tool information overlay at the position of the working end of the first tool in the image of the work site, wherein the first tool information overlay visually indicates an identity of the first input device.

22. The system of claim 21, wherein the first input device is a left input device, and wherein the first tool information overlay is a letter “L”.

23. The system of claim 21, wherein the first input device is a right input device, and wherein the first tool information overlay is a letter “R”.

24. The system of claim 21, wherein the first tool information overlay further visually indicates an identity of the first operator.

25. The system of claim 24, wherein the first tool information overlay includes a symbol or a color associated with the first operator.

26. The system of claim 21, further comprising a second input device communicatively coupled to the first robotic arm, the second input device being manipulatable by a second operator.

27. The system of claim 26, wherein when the first input device is operatively associated with the first robotic arm, the first tool information overlay is displayed in a first color associated with the first operator.

28. The system of claim 27, wherein when the second input device is operatively associated with the first robotic arm, the first tool information overlay is displayed in a second color associated with the second operator.

29. The system of claim 28, wherein the processor is further configured to:
change a display of the first tool information overlay from the first color to the second color when the first robotic arm changes from being operatively associated with the first input device to being operatively associated with the second input device.

30. The system of claim 21, further comprising:
a second robotic arm operatively coupleable to a second tool, the second tool comprising a working end; and
a second input device communicatively coupled to the second robotic arm, the second input device being manipulatable by the first operator,
wherein the image of the work site further includes an image of the working end of the second tool, and
wherein the processor is further configured to:
determine a position of the working end of the second tool in the image of the work site; and
render a second tool information overlay at the position of the working end of the second tool in the image of the work site, wherein the second tool information overlay visually indicates an identity of the second input device.

31. The system of claim 21, wherein the first tool information overlay is displayed over an end effector of the working end of the first tool.

32. The system of claim 21, wherein the first tool information overlay is displayed over a portion of the working end of the first tool proximal of an end effector.

33. A method comprising:
displaying an image of a work site to be displayed on a display, the image captured by an image capture device from a perspective of an image reference frame, the image of the work site including an image of a working end of a first tool operatively coupled to a first robotic arm;
determining a position of the working end of the first tool in the image of the work site; and
rendering a first tool information overlay at the position of the working end of the first tool in the image of the work site, wherein the first tool information overlay visually indicates an identity of a first input device manipulatable by a first operator, the first input device communicatively coupled to the first robotic arm.

34. The method of claim 33, wherein the first input device is a left input device, and wherein the first tool information overlay is a letter “L”.

35. The method of claim 33, wherein the first input device is a right input device, and wherein the first tool information overlay is a letter “R”.

36. The method of claim 33, wherein the first tool information overlay further visually indicates an identity of the first operator.

37. The method of claim 33, wherein the first tool information overlay further visually indicates an identity of a second input device manipulatable by a second operator, the second input device communicatively coupled to the first robotic arm.

38. The method of claim 37, wherein when the first input device is operatively associated with the first robotic arm, the first tool information overlay is displayed in a first color associated with the first operator.

39. The method of claim 38, wherein when the second input device is operatively associated with the first robotic arm, the first tool information overlay is displayed in a second color associated with the second operator.

40. The method of claim 39, further comprising:
changing a display of the first tool information overlay from the first color to the second color when the first robotic arm changes from being operatively associated with the first input device to being operatively associated with the second input device.
